(19) United States
(12) Patent Application Publication
(10) Pub. No.: US 2015/0209252 A1
(43) Pub. Date: Jul. 30, 2015

(54) Heat Treated Silica for Improved Dentifrice

(71) Applicant: J.M. Huber Corporation, Atlanta, GA (US)

(72) Inventor: Patrick McGill, Darlington, MD (US)

(73) Assignee: J.M. Huber Corporation

(21) Appl. No.: 14/604,828

(22) Filed: Jan. 26, 2015

Related U.S. Application Data
(60) Provisional application No. 61/931,855, filed on Jan. 27, 2014.

(51) Int. Cl. A61K 8/25; A61K 8/81; A61Q 11/00; A61K 8/22

(52) U.S. Cl. CPC A61K 8/25; A61K 8/22; A61K 8/8176; A61Q 11/00; A61K 2800/80

(57) ABSTRACT

Heat treated silica materials are disclosed, together with methods of making such materials, and dentifrice compositions containing the heat treated silica materials. The heat treated silica materials can contain trace amounts of cristobalite, typically less than 1 wt. %, and the heat treated silica materials often have a loss on ignition of less than about 2 wt. %.

### REFERENCE TO RELATED APPLICATION

This application claims the benefit of U.S. Provisional Application Ser. No. 61/931,855, filed on Jan. 27, 2014, the disclosure of which is incorporated herein by reference in its entirety.

### TECHNICAL FIELD

The present disclosure relates to silica materials, and specifically to heat treated silica materials that can provide improved stability with dentifrice compositions.

### BACKGROUND

Conventional dentifrice compositions comprise an abrasive substance to assist in the removal of dental deposits. A dentifrice should ideally be sufficiently abrasive to clean the tooth surface, but not so abrasive as to damage the hard tissues of the tooth. Dentifrice compositions also frequently comprise a whitening formulation to further assist in the removal of dental deposits, such as a pellicle film, and to improve tooth aesthetics. For example, and without limitation, one whitening formulation comprises a polyvinylpyrrolidone-hydrogen peroxide complex.

The performance of a dentifrice can thus be highly sensitive to the aggressiveness of the abrasive substance and its compatibility with the whitening formulation. Synthetic low-structure silica materials have frequently been used as abrasive substances due to their effectiveness as abrasives as well as their low toxicity characteristics and compatibility with other dentifrice components, such as sodium fluoride.

To date, conventional abrasive materials have limitations associated with maximizing cleaning and minimizing dentin abrasion. Additionally, many conventional abrasive materials have limited compatibility with whitening formulations and can facilitate or catalyze the decomposition of the whitening complexes, resulting in poor dentifrice tube stability. Accordingly, there exists a need to develop new dental abrasives and dentifrice materials that exhibit high cleaning properties, have acceptable dentin abrasion levels, and have high compatibility with whitening formulations. This need and other needs are satisfied by the compositions and methods of the present disclosure.

### SUMMARY

In accordance with the purpose(s) of the invention, as embodied and broadly described herein, this disclosure, in one aspect, relates to silica materials, and specifically to heat treated silica materials that can provide improved stability with dentifrice compositions.

In one aspect, the present disclosure provides a dentifrice comprising a precipitated silica material, wherein the precipitated silica material has been heated at a temperature and for a period of time sufficient to remove all or a portion of water from the surface thereof.

In another aspect, the present disclosure provides a dentifrice comprising a polyvinylpyrrolidone-hydrogen peroxide complex and a precipitated silica material, wherein the precipitated silica material has been heated at a temperature and for a period of time sufficient to at least partially dehydroxylate the surface thereof.

In another aspect, the present disclosure provides a dentifrice comprising a polyvinylpyrrolidone-hydrogen peroxide complex and a precipitated silica material, wherein the precipitated silica material has no or substantially no free or chemically bound water on a surface thereof.

In another aspect, the present disclosure provides a dentifrice composition, wherein a portion of the precipitated silica material is crystalline.

In another aspect, the present disclosure provides a dentifrice composition comprising a polyvinylpyrrolidone-hydrogen peroxide complex and a precipitated silica material, wherein the precipitated silica material comprises from greater than 0 to less than about 1 wt. % cristobalite, and the precipitated silica material is characterized by a loss on ignition of less than about 2 wt. %.

In another aspect, the present disclosure provides a dentifrice composition, wherein no more than 0.1 wt. % of the precipitated silica material comprises cristobalite.

In another aspect, the present disclosure provides a dentifrice composition, having improved stability as compared to a dentifrice comprising a polyvinylpyrrolidone-hydrogen peroxide complex and a conventional non-heat treated precipitated silica material.

In another aspect, the present disclosure provides a method for preparing a dentifrice composition, the method comprising heat treating a precipitated silica material at a temperature and for a period of time sufficient to: a) remove all or a portion of water from the precipitated silica material without changing the structure thereof; b) at least partially dehydroxylate the surface of the precipitated silica material; or a combination thereof, and then contacting the precipitated silica material with a polyvinylpyrrolidone-hydrogen peroxide complex.

In another aspect, the present disclosure provides a method for preparing a dentifrice composition, the method comprising (a) heat treating a precipitated silica material at a temperature and for a period of time sufficient to form a heat treated silica material comprising from greater than 0 to less than about 1 wt. % cristobalite, the heat treated silica material characterized by a loss on ignition of less than about 2 wt. %; and (b) contacting the heat treated silica material with a polyvinylpyrrolidone-hydrogen peroxide complex and other components to form the dentifrice composition.

In another aspect, the present disclosure provides a precipitated silica material comprising from greater than 0 to less than about 1 wt. % cristobalite (e.g., from greater than 0 to less than about 0.1 wt. % cristobalite), and characterized by a loss on ignition of less than about 2 wt. % (e.g., less than about 1 wt. %).

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

FIG. 1 illustrates Glycerine Refractive Index Curves for heat treated silica materials.

FIG. 2 illustrates Sorbitol Refractive Index Curves for heat treated silica materials.

FIG. 3 illustrates tube stability of dentifrice compositions with silica materials subjected to various heat treatment conditions.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which this invention belongs.

As used herein, unless specifically stated to the contrary, the singular forms “a,” “an” and “the” include plural referents unless the context clearly dictates otherwise. Thus, for example, reference to “a filler” or “a solvent” includes mixtures of two or more fillers, or solvents, respectively.

As used herein, the terms “optional” or “optionally” means that the subsequently described event or circumstance can or cannot occur, and that the description includes instances where said event or circumstance occurs and instances where it does not.

For purposes of this invention, a “dentifrice” has the meaning defined in Oral Hygiene Products and Practice, Morton Pader, Consumer Science and Technology Series, Vol. 6, Marcel Dekker, NY 1988, p. 200, which is incorporated herein by reference. Namely, a “dentifrice” is “ . . . a substance used with a toothbrush to clean the accessible surfaces of the teeth. Dentifrices are primarily composed of water, detergent, humectant, binder, flavoring agents, and a finely powdered abrasive as the principal ingredient . . . a dentifrice is considered to be an abrasive-containing dosage form for delivering anti-caries agents to the teeth.” Dentifrice formulations contain ingredients which must be dissolved prior to incorporation into the dentifrice formulation (e.g. anti-caries agents such as sodium fluoride, sodium phosphates, flavoring agents such as saccharin). In another aspect, a dentifrice can comprise other oral delivery methods and/or articles of an oral care agent, such as, for example, including a gum, a tablet, a strip, a dissolving strip, or a combination thereof.

The Brass Einlehner (BE) Abrasion test used to measure the hardness of the precipitated silicas/silica gels reported in this application is described in detail in U.S. Pat. No. 6,616,916, incorporated herein by reference, and involves an Einlehner AT-1000 Abrader generally used as follows: (1) a Fourdrinier brass wire screen is weighed and exposed to the action of a 10% aqueous silica suspension for a fixed length of time; (2) the amount of abrasion is then determined as milligrams brass lost from the Fourdrinier wire screen per 100,000 revolutions. The result, measured in units of mg loss, can be characterized as the 10% brass Einlehner (BE) abrasion value.

The Radioactive Dentin Abrasion (RDA) values of dentifrices containing the silica compositions used in this invention are determined according to the method set forth by Hefferen, Journal of Dental Res., July-August 1976, 55 (4), pp. 563-573, and described in Wason U.S. Pat. Nos. 4,340,583, 4,420,312 and 4,421,527, which publications and patents are incorporated herein by reference.

The cleaning property of dentifrice compositions is typically expressed in terms of Pellicle Cleaning Ratio (“PCR”) value. The PCR test measures the ability of a dentifrice composition to remove pellicle film from a tooth under fixed brushing conditions. The PCR test is described in “In Vitro Removal of Stain with Dentifrice,” G. K. Stookey, et al., J. Dental Res., 61, 1236-9, 1982. Both PCR and RDA results vary depending upon the nature and concentration of the components of the dentifrice composition. PCR and RDA values are unitless.

The terms “Loss on Drying” or “LOD,” unless specifically stated to the contrary, are intended to refer to the amount of volatile matter driven off upon heating a sample for 2 hours at 105° C. In one aspect, LOD can be expressed as % LOD. In other aspects, LOD can be defined according to the Food Chemicals Codex 7^{th} Edition, General Tests and Assays, Appendix IIC, “Loss on Drying”, and current Silicon Dioxide, Sodium Aluminosilicate, and Calcium Silicate monographs; or the United States Pharmacopeia 33—National Formulary 28 supplement 1 Reissue, effective Oct. 1, 2010, Physical Tests and Determinations General Chapter <731> Loss on Drying; and current Dental-Type Silica and Calcium Silicate monographs, both of which are hereby incorporated by reference for the purpose of describing loss on drying the methodology to determine the same.

The terms “Loss on Ignition” or “LOI,” unless specifically stated to the contrary, are intended to refer to the loss in weight of a silica or silicate pigment upon ignition and/or heating at 900° C. for 2 hours, after previously drying at 105° C. for 2 hours. In one aspect, a sample can be dried and then cooled to room temperature, for example, in a dessicator, and then be heated in a muffle furnace at 900° C. for 2 hours. In another aspect, LOI can be expressed as % LOI.

The term “CTAB” refers to the surface area of silica as measured using the CTAB (Cetyltrimethylammonium Bromide) Surface Area Test Method (ASTM D6845-12).

The term “BET” refers to a measurement of the specific surface area of silica as defined by Brunauer-Emmett-Teller (BET) theory (S. Brunauer, P. H. Emmett and E. Teller, J. Am. Chem. Soc., 1938, 60, 309).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present disclosure provides silica materials that can be used in dentifrice compositions, methods for the preparation thereof, and dentifrice compositions comprising the inventive silica materials. It would be desirable to have dentifrice materials that exhibit good cleaning properties and are also compatible with other components of the dentifrice composition, such as, for example, whitening components. In various aspects, the present disclosure provides silica materials that exhibit improved compatibility with whitening systems, such as, for example, polyvinylpyrrolidone-hydrogen peroxide compositions. In another aspect, the present disclosure provides dentifrice compositions comprising such improved silica materials.

### Silica

Silica materials suitable for use in dentifrice compositions can comprise synthetically produced, precipitated silicas. In one aspect, the silica material can be a low-structure silica material. These silica materials can be produced using various procedures.

In various aspects, a suitable alkali metal silicate can be used with the methods described herein to prepare a silica material. In one aspect a water soluble silicate, such as, for example, a potassium silicate, a sodium silicate, or a combination thereof, can be used. In other aspects, a silicate compound having a desirable silicate:metal molar ratio (MR) can be selected. For example, sodium silicates can generally have a silicate:metal molar ratio of from about 1:1 to about 4:1. In one aspect, the silicate compound can have a molar ratio of from about 1:1 to about 4:1, for example, about 1:1, 1.25:1, 1.5:1, 1.75:1, 2:1, 2.25:1, 2.5:1, 2.75:1, 3:1, 3.25:1, 3.5:1, 3.6:1, 3.7:1, 3.8:1, 3.9:1, or 4:1; or from about 2.5:1 to about 3.8:1, for example, about 2.5:1, 2.75:1, 3:1, 3.25:1, 3.5:1, 3.75:1, or 3.8:1. In another aspect, the silicate compound can have a molar ratio of about 3.32:1.

In one aspect, the silicate compound, such as, for example, sodium silicate, can be dissolved in water to produce a silicate solution. In one aspect, the solution comprises from about 8 wt. % to about 35 wt. % silicate, for example, about 8, 9, 11, 13, 15, 17, 19, 21, 23, 25, 27, 29, 31, 33, or 35 wt. % silicate. In another aspect, the solution comprises from about 8 wt. % to about 20 wt. % silicate, for example, about 8, 10, 12, 14, 16, 18, or 20 wt. % silicate. In a specific aspect, the silicate solution can comprise about 19.5 wt. % silicate. In other aspects, the resulting silicate solution can have a silicate concentration less than or greater than any value specifically recited herein, and the present disclosure is intended to cover such solutions. In still other aspects, silicate solutions are commercially available and can be purchased and utilized as-is (e.g., from Sigma-Aldrich Corporation, St. Louis, Mo., USA).

In another aspect, the silicate solution can have a silicate concentration of from about 2 wt. % to about 10 wt. %, for example, about 2, 2.5, 3, 3.5, 4, 4.5, 5, 5.5, 6, 6.5, 7, 7.5, 8, 8.5, 9, 9.5, or 10 wt. %. In yet another aspect a silicate solution having a higher concentration, for example, about 20 wt. %, can be diluted in water to a lower concentration as described herein. For example, a quantity of a 19.5 wt. % silicate solution can be diluted to a concentration of about 5.5 wt. %.

The silicate solution can optionally be heated, for example, to about 75° C., about 80° C., about 85° C., about 87° C., about 90° C., or higher, and/or stirred.

The silicate solution and an acidulating agent, such as, for example, sulfuric acid, can then be contacted. In various aspects, the acid can comprise nitric acid, hydrochloric acid, phosphoric acid, boric acid, hydrofluoric acid, sulfuric acid, or a combination thereof. In one aspect, the acid can comprise a mineral acid. In another aspect, the acid comprises sulfuric acid. In yet another aspect, the acid can comprise a mixture of two or more individual acids. In other aspects, other suitable acids can be utilized in addition to or in lieu of any acid specifically recited herein. The concentration and/or pH of the acidulating agent can be any concentration and/or pH suitable for use in preparing a precipitated silica material. In various aspects, the acidulating agent can comprise from about 5 wt. % to about 35 wt. % sulfuric acid, for example, about 5, 7, 9, 11, 13, 15, 17, 19, 21, 23, 25, 27, 29, 31, 33, or 35 wt. %; or from about 12 wt. % to about 22 wt. % sulfuric acid, for example, about 12, 13, 14, 15, 16, or 17 wt. % sulfuric acid.

The silicate solution and acidulating agent can be added to the vessel over a period of time. In another aspect, the silicate solution and the acidulating agent can be added simultaneously or substantially simultaneously. In various aspects, the addition ratio of silicate solution to acidulating agent can be about from about 1:0.1 to about 1:0.6, for example, about 1:0.1, 1:0.15, 1:0.2, 1:0.25, 1:0.3, 1:0.33, 1:0.35, 1:0.4, 1:0.45, 1:0.5, 1:0.55, 1:0.6. In another aspect, the addition ratio of silicate solution to acidulating agent can be about 1:0.33.

The silicate solution and acidulating agent can be added for a fixed period of time or until exhausted. In one aspect, addition of the silicate solution can be stopped after a period of time, wherein addition of the acidulating agent continues for an additional period of time. In one aspect, the addition of the acidulating agent can be continued until a desired pH is reached in the reaction vessel. In such an aspect, the acidulating agent can be added until the pH in the reaction vessel is from about 4.5 to about 6.5, for example, about 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, or 6.5; or from about 5.3 to about 5.7, for example, about 5.3, 5.4, 5.5, 5.6, or 5.7.

In another aspect, addition of the silicate solution and/or acidulating agent can be stopped at any desired time and the pH of the reaction vessel subsequently adjusted to a desired value.

After contacting the silicate solution and the acidulating agent, the resulting precipitated silica slurry can be allowed to digest for a period of time. In one aspect, the precipitated silica slurry can be allowed to digest at a temperature of about 90° C. for a period of at least about 10 minutes. In other aspects, a digestion step, if performed, can be performed for any suitable length of time and at any suitable temperature, and one of skill in the art, in possession of this disclosure, could readily determine appropriate digestion conditions. After digestion, the resulting precipitated silica material can be separated, for example, by filtration, from the solution. The separated silica material can optionally be washed to remove all or a portion of the acid and any unreacted, dissolved silicate. In one aspect, the separated silica material can be washed, for example, with deionized water, until a conductivity of about 1,500 μS is reached. In other aspects, the separated silica material can be utilized as-is, or can be washed to a greater or lesser extent that that specifically described herein. In another aspect, the precipitated silica material can be dried, for example, by placing in a 105° C. oven overnight. In another aspect, the precipitated silica material can be spray dried.

If desired, the precipitated silica material can optionally be processed to achieve a desired average particle size or particle size distribution. In various aspects, the precipitated silica can be milled and/or ground to a desired average particle size, for example, of about 10 μm.

In one aspect, the reaction (e.g., contacting) of the silicate solution and acidulating agent can be conducted at an elevated temperature and/or while stirring so as to avoid the formation of a gel or aggregation of silica particles. In other aspects, it should be understood that the method of contacting and/or mixing, concentration and addition rates of reactants, temperature, and pH can each affect the properties of the resulting precipitated silica.

In one aspect, the preparation of a precipitated silica can be conducted as described in one or more of U.S. Pat. Nos. 2,739,073, 2,848,346, and 5,891,421, which are hereby incorporated by reference in their entirety for the purpose of disclosing methods for preparing precipitated silica materials. In other aspects, one of skill in the art, in possession of this disclosure, could readily determine appropriate reactants and reaction conditions to prepare a desired precipitate silica. In another aspect, the process to prepare a precipitated silica as described herein can be performed in a batch process, a semi-continuous process, or a continuous process. In one aspect, all or a portion of the steps are performed in a batch process. In another aspect, the process can be at least partially continuous, wherein a silicate solution and an acidulating agent can be continuously fed into a loop reaction zone, wherein at least a portion of the acidulating agent and silicate react to form a precipitated silica. In one aspect, the precipitated silica material can comprise a silica suitable for use in a dentifrice composition, such as, for example, a Zeodent® silica, available from J.M. Huber Corporation (Havre de Grace Md., U.S.A). In various aspects, the precipitated silica material can comprise Zeodent® 105 silica, Zeodent® 114 silica, Zeodent® 115 silica, Zeodent® 165 silica, or a combination thereof, each of which are available from J.M. Huber Corporation (Havre de Grace Md., U.S.A). In still other aspects, the precipitated silica material can comprise a precipitated silica material not specifically recited herein, and the present disclosure is not intended to be limited to any particular silica material.

In one aspect, the precipitated silica of the present disclosure does not comprise a metal adduct or other metal (e.g., a transition metal or other metal, such as aluminum, zinc, tin, strontium, iron, copper, and the like). In another aspect, the precipitated silica of the present disclosure does not comprise a metal adduct or other metal, other than any residual metal present from an original silicate compound used in the preparation of the silica material. In yet another aspect, the precipitated silica of the present disclosure contains substantially no (less than 0.5 wt. % of a) transition metal or other metal, either singly or in combination. In still another aspect, the precipitated silica of the present disclosure can contain less than 0.5 wt. %, less than 0.25 wt. %, less than 0.1 wt. %, less than 0.075 wt. %, less than 0.05 wt. %, or less than 0.02 wt. %, of any individual transition metal or other metal excluding aluminum and sodium; additionally or alternatively, the precipitated silica of the present disclosure can contain less than 0.5 wt. %, less than 0.25 wt. %, or less than 0.15 wt. % of aluminum; additionally or alternatively, the precipitated silica of the present disclosure can contain less than 1.5 wt. %, less than 1 wt. %, or less than 0.5 wt. % of sodium.

### Heat Treatment

In one aspect, the precipitated silica material can be heat treated as described herein. In various aspects, a heat treated silica material can exhibit a reduced content of free and/or chemically bound water. In another aspect, the precipitated silica material can exhibit improved stability in a dentifrice composition comprising a peroxide composition, such as, for example, a polyvinylpyrrolidone-hydrogen peroxide composition.

The specific time and temperature at which a precipitated silica can be heated can vary. In another aspect, the precipitated silica can be heated for a time and at a temperature sufficient to remove all or a portion of free and/or chemically bound water that can be present on the surface of the silica material. In another aspect, the heat treatment can at least partially dehydroxylate the silica surface without significantly changing the structure of the silica material. In yet another aspect, the heat treatment can dehydroxylate all or a portion of the silica surface with no or substantially no change to the structure of the silica material. In one aspect, the heat treatment is sufficient to remove all or a portion of water bound to the silica surface, but does not crystallize the silica material.

In one aspect, a precipitated silica material can be heated at a temperature and for a time to convert the precipitated silica, or a portion thereof, from an amorphous phase to a crystalline phase. In another aspect, the silica material can be heated at a temperature and for a time such that a portion of the heat treated silica, for example, from about 0.1 wt. % to about 80 wt. %, comprises a crystalline phase. In another aspect, the silica material can be heated at a temperature and for a time such that from about 0.1 wt. % to about 80 wt. %, for example, about 0.1, 0.5, 1, 2, 3, 4, 5, 8, 10, 30, 40, 50, 60, 70, or 80 wt. % of the heat treated material comprises a crystalline phase.

In another aspect, the silica material can be heated at a temperature and for a time such that a portion of the heat treated silica material comprises cristobalite and/or tridymite. In another aspect, the silica material, after heat treating, can comprise up to, for example, about 1 wt. % cristobalite and/or tridymite. In another aspect, the silica material, after heat treating, can comprise up to, for example, about 0.5 wt. % cristobalite and/or tridymite. In another aspect, the silica material, after heat treating, can comprise up to, for example, about 0.1 wt. % cristobalite and/or tridymite. In another aspect, the silica material, after heat treating, can comprise up to, for example, about 0.05 wt. % cristobalite and/or tridymite. In still another aspect, the silica material, after heat treating, can comprise up to, for example, a trace amount of cristobalite. In still another aspect, the silica material, after heat treating, can comprise up to, for example, a trace amount of tridymite. Accordingly, the silica material, after heat treating, can comprise from greater than 0 to less than about 1 wt. % cristobalite; alternatively, from greater than 0 to less than about 0.5 wt. % cristobalite; alternatively, from greater than 0 to less than about 0.25 wt. % cristobalite; or alternatively, from greater than 0 to less than about 0.1 wt. % cristobalite.

In one aspect, the precipitated silica can be heated at a temperature of from about 650° C. to about 1,000° C., for example, about 650, 675, 700, 725, 750, 775, 800, 825, 850, 875, 900, 925, 950, 975, or 1,000° C. In another aspect, the precipitated silica can be heated at a temperature of from about 700° C. to about 950° C., for example about 700, 725, 750, 775, 800, 825, 850, 875, 900, 925, or 950° C. In yet another aspect, the precipitated silica can be heated at a temperature of from about 700° C. to about 800° C., for example about 750, 775, or 800; or from about 750° C. to about 850°, for example, about 750, 775, 800, 825, or 850° C.

The length of time that a silica material is heated can also vary, for example, depending upon the specific temperature employed. In various aspects, a silica material can be heated for a period of time ranging from about 5 minutes to about 1200 minutes or more, for example, about 5, 10, 20, 30, 40, 50, 60, 90, 120, 150, 180, 210, 240, 270, 300, 330, 360, 390, 420, 450, 480, 510, 540, 570, 600, 630, 660, 690, 720, 750, 780, 810, 840, 870, 900, 930, 960, 990, 1020, 1050, 1080, 1110, 1140, 1170, 1200 minutes, or more. In another aspect, a silica material can be heated for a period of time from about 20 minutes to about 60 minutes, for example, about 20, 30, 40, 50, or 60 minutes. In another aspect, a silica material can be heated for a period of time from about 120 minutes to about 960 minutes, for example, about 120, 240, 480, or 960 minutes. In one aspect, the silica material can be heated for a period of from about 10 to about 20 hours, for example, about 10, 11, 12, 13, 14, 15, 16, 17, 18, 19, or 20 hours; from about 14 to about 18 hours, for example, about 14, 15, 16, 17, or 18 hours; or from about 15 to about 17 hours, for example, about 15, 15.5, 16, 16.5, or 17 hours. In one aspect, the precipitated silica material can be heat treated for a period of about 16 hours.

It should be appreciated that the time needed to sufficiently treat a silica material can vary depending on the temperature at which the material is heated. In one aspect, a precipitated silica material can be heat treated at a temperature of about 550° C. for a period of time, for example, from about 20 minutes to about 960 minutes or more. In another aspect, a precipitated silica material can be heat treated at a temperature of about 650° C. for a period of time, for example, from about 20 minutes to about 960 minutes or more. In another aspect, a precipitated silica material can be heat treated at a temperature of about 750° C. for a period of time, for example, from about 20 minutes to about 960 minutes. In one aspect, a precipitated silica material can be heat treated at a temperature of about 750° C. for about 480 minutes to about 960 minutes or more. In yet another aspect, a precipitated silica material can be heated at a temperature of about 750° C. for a period of about 16 hours. In one aspect, a precipitated silica material can be heated for a period of time sufficient to remove all or a portion of free and/or chemically bound water from the silica material and/or to at least partially dehydroxylate the surface of the silica material. In another aspect, a precipitated silica material can be heated at a temperature of about 850° C. for a period of from about 20 minutes to about 960 minutes or more. In still another aspect, a precipitated silica material can be heated at a temperature of about 850° C. for a period of at least about 120 minutes, for example, from about 120 to about 960 minutes, from about 120 to about 480 minutes, or from about 120 to about 240 minutes. In yet another aspect, a precipitated silica material can be heated at a temperature of about 950° C. for a period of at least about 20 minutes, for example, from about 20 minutes to about 960 minutes, from about 20 minutes to about 480 minutes, from about 20 minutes to about 240 minutes, or from about 20 minutes to about 120 minutes. In still other aspects, a silica material can be heated for a time less than or greater than any time specifically recited herein.

It should also be noted that the specific temperature and time at which a silica material is heated can depend upon, for example, the size of the sample, the volume of the sample, and/or the container in which the sample is placed while heating. In one aspect, the conditions needed to produce a desirable heat treated silica material can be dependent upon the amount and rate of heat transfer to a sample. Thus, a small sample can be effectively treated at a lower temperature and/or at a shorter time than a similar large sample. Similarly, a sample that is spread out, for example, having a higher ratio of external surface area (i.e., of the total sample) to volume, can be effectively heated in a shorter amount of time and/or at a lower temperature than a comparable bulk sample having a lower ratio of external surface area to volume. In another aspect, a sample disposed in a thermally conductive container while heating can be effectively heated for a shorter period of time and/or at a lower temperature than a comparable sample disposed in a thermally insulating container. Impurities in the starting silica material can also impact the heating conditions required to produce a desirable heat treated silica material. One of skill in the art, in possession of this disclosure, could readily determine appropriate heating conditions (e.g., time, temperature, sample size, container) to produce a desirable heat treated silica material.

In one aspect, a precipitated silica material can be placed in a room temperature oven or furnace, while the temperature of the oven or furnace is raised to the desired temperature, for example, about 750° C. The atmosphere of the heating environment can vary, but can comprise air, an inert atmosphere, or a mixture of gases. In a specific aspect, the heating atmosphere comprises an air environment. In another aspect, the precipitated silica material, after heating, can be removed from the oven or furnace, or it can be allowed to cool as the oven or furnace is cooled, for example, over a period of from about 2 to about 4 hours.

In one aspect, once removed from the oven or furnace, or once cooled, the heat treated precipitated silica material can be placed in a dessicator or storage container to minimize and/or prevent the absorption of moisture.

### Heat Treated Silica Material

In one aspect, the heat treated precipitated silica material exhibits a reduced level of free and/or chemically bound water. In another aspect, the heat treated precipitated silica material exhibits a reduced affinity for water. For example, upon exposure to moisture, the heat treated precipitated silica material can absorb less water than a comparable silica material not heat treated in accordance with the present disclosure.

In another aspect, the heat treated precipitated silica material remains an amorphous or substantially amorphous material, for example, having less than about 5% crystallinity. In another aspect, the heat treated precipitated silica material can comprise an amorphous structure with a trace of cristobalite. In yet another aspect, the heat treated precipitated silica material can comprise from greater than 0 to less than about 1 wt. % cristobalite; alternatively, from greater than 0 to less than about 0.5 wt. % cristobalite; alternatively, from greater than 0 to less than about 0.25 wt. % cristobalite; or alternatively, from greater than 0 to less than about 0.1 wt. % cristobalite. In still another aspect, none or substantially none of the silica material comprises cristobalite.

The heat treated precipitated material ideally comprises less than about 1 wt. % water, or less than about 0.75 wt. % water, as determined by Loss on Drying (LOD) techniques. In another aspect, the heat treated precipitated silica material exhibits a pre-dried Loss on Ignition (LOI) of less than about 2 wt. %, less than about 1.5 wt. %, less than about 1 wt. %, or less than about 0.75 wt. %.

In various aspects, Brass Einlehner (BE) Abrasion values of the resulting silica material, after heat treatment, can be greater than about 5 mg loss/100,000 revolutions, greater than 5.5 mg loss/100,000 revolutions, or greater than about 6 mg loss/100,000 revolutions. In another aspect, the Brass Einlehner (BE) Abrasion values of the resulting silica material can be from about 4 to about 20 mg loss/100,000 revolutions, from about 5 to about 15 mg loss/100,000 revolutions, from about 5 to about 10 mg loss/100,000 revolutions, from about 4 to about 8 mg loss/100,000 revolutions, or from about 5 to about 7 mg loss/100,000 revolutions. In other aspects, the Brass Einlehner (BE) Abrasion values can increase to about 10, about 15, about 20, about 25, about 30, or more, depending upon the specific silica material utilized and the heat treatment conditions to which the sample is subjected.

Generally, the BET surface area of suitable heat treated silica materials contemplated herein can fall within a range from about 20 to about 60 m²/g, from about 20 to about 55 m²/g, from about 25 to about 60 m²/g, from about 25 to about 55 m²/g, or from about 30 to about 50 m²/g. Additionally or alternatively, suitable heat treated silica materials can be characterized by an oil absorption in a range from about 70 to about 120 cc/100 g, from about 70 to about 115 cc/100 g, from about 75 to about 120 cc/100 g, from about 75 to about 115 cc/100 g, or from about 80 to about 110 cc/100 g.

### Dentifrice Composition

The inventive precipitated silica materials can be ready-to-use additives in the preparation of oral cleaning compositions, such as dentifrices, toothpastes, and the like. In one aspect, the heat treated precipitated silica material can be combined with one or more dentifrice components, such as, for example, abrasives, rheological aids, whiteners, sweeteners, flavoring additives, surfactants, colorants, or other components to form a dentifrice composition. If combined with other abrasives (such as any of the products offered by J. M. Huber Corporation under the trade name ZEODENT®), such an abrasive may be added in any amount. In one aspect, the inventive silica material can be used at a loading of about 20 wt. % in the dentifrice composition. In other aspects, the inventive silica material can be used in excess of 20% and up to about 25 wt. %, 30 wt. %, 35 wt. % or more. In one aspect, the inventive heat treated precipitated silica material can be used to replace all or a portion of another typical dentifrice component, such as, for example, calcium pyrophosphate. In such an aspect, a reduced amount of the inventive silica can be used to replace a larger portion of calcium pyrophosphate. For example, a dentifrice comprising about 20 wt. % of the inventive heat treated precipitated silica material can provide similar or improved performance as compared to a conventional dentifrice comprising about 35-45 wt. % calcium pyrophosphate.

The inventive silica material can be utilized alone as the cleaning agent component in a dentifrice compositions or in combination with one or more other materials. Thus, a combination of the inventive materials with other materials physically blended therewith within a suitable dentifrice formulation can be useful to accord targeted dental cleaning and abrasion results at a desired protective level. Thus, any number of other conventional types of dentifrice additives, such as, for example, abrasives, can be present within inventive dentifrices in accordance with this invention. Other such materials can include, for example, and without limitation, precipitated calcium carbonate (PCC), ground calcium carbonate (GCC), dicalcium phosphate or its dihydrate forms, perlite, titanium dioxide, calcium pyrophosphate, hydrated alumina, calcined alumina, insoluble sodium metaphosphate, insoluble potassium metaphosphate, insoluble magnesium carbonate, zirconium silicate, aluminum silicate, and so forth, and can be introduced within the desired abrasive compositions to tailor the polishing characteristics of the target formulation (dentifrices, for example, etc.), if desired, as well. In another aspect, the heat treated silica material or a portion of a dentifrice containing the heat treated silica material can be combined with one or more other heat treated materials, such as, for example, a different heat treated silica material and/or a heat treated silica gel.

In one aspect, the dentifrice composition can comprise one or more whitening components, such as, for example, a polyvinylpyrrolidone-hydrogen peroxide (PVP/H₂O₂) complex (available from Ashland Chemicals, Smyrna, Ga., U.S.A.). Such PVP/H₂O₂ complexes can release hydrogen peroxide upon contact with water or saliva in the mouth of a subject. When combined with a PVP/H₂O₂ composition, a conventional precipitated silica material can act as a decomposition catalyst. While not wishing to be bound by theory, it is believed that hydrogen peroxide is bound to polyvinylpyrrolidone via hydrogen bonding, and that water, such as, for example, free and/or chemically bound water present on the surface of a conventional precipitated silica material, can interfere with hydrogen bonding. Thus, water present on the surface of a conventional precipitated silica material can act in a manner similar to water or saliva in a subject's mouth, resulting in decomposition of the complex and release of hydrogen peroxide. The stability of a dentifrice comprising a PVP/H₂O₂ complex and a precipitated silica material can be affected (i.e., reduced) by the water, for example, free and/or chemically bound water, present on the surface of the precipitated silica material.

In one aspect, the inventive heat treated precipitated silica material exhibits a reduced water content and has a lower affinity for water than a comparable conventional silica material. The reduced water content can minimize and/or eliminate decomposition of the hydrogen peroxide in a polyvinylpyrrolidone-hydrogen peroxide complex and improve stability of a resulting dentifrice composition. While not wishing to be bound by theory, it is believed that the heat treatment methods described herein can at least partially dehydroxylate the surface of a precipitated silica material. In one aspect, such heat treatment and/or dehydroxylation can improve the stability of a resulting silica-PVP/H₂O₂ complex, even upon contact with water. In another aspect, and while not wishing to be bound by theory, decomposition of a PVP/H₂O₂ complex with non-heat treated silica materials is not due solely to the addition of water. In such an aspect, the surface activity (e.g., free moisture and hydroxyl group concentration) of a silica material can affect the stability of a PVP/H₂O₂ complex.

In addition, as noted above, the inventive silica material can be used in conjunction with other materials, such as dicalcium phosphate, dicalcium phosphate dihydrate, calcium metasilicate, calcium pyrophosphate, alumina, calcined alumina, aluminum silicate, precipitated and ground calcium carbonate, chalk, bentonite, particulate thermosetting resins and other suitable abrasive materials known to a person of ordinary skill in the art.

In addition to the abrasive component, a dentifrice can optionally comprise one or more organoleptic enhancing agents. Organoleptic enhancing agents include humectants, sweeteners, surfactants, flavorants, colorants and thickening agents (also sometimes known as binders, gums, or stabilizing agents). Humectants serve to add body or “mouth texture” to a dentifrice as well as prevent the dentifrice from drying out. Suitable humectants can comprise polyethylene glycol (at a variety of different molecular weights), propylene glycol, glycerin (glycerol), erythritol, xylitol, sorbitol, mannitol, lactitol, and hydrogenated starch hydrolyzates, as well as mixtures of these compounds. Typical levels of humectants, if present, can range from about 20 wt. % to about 30 wt. % of a dentifrice composition.

Sweeteners can be added to a dentifrice composition to impart a pleasing taste to the product. Suitable sweeteners include saccharin (as sodium, potassium or calcium saccharin), cyclamate (as a sodium, potassium or calcium salt), acesulfane-K, thaumatin, neohisperidin dihydrochalcone, ammoniated glycyrrhizin, dextrose, levulose, sucrose, mannose, and glucose.

In one aspect, surfactants can also be used in a dentifrice composition to make the composition more cosmetically acceptable. A surfactant, if used, can be a detersive material which imparts to the composition detersive and foaming properties. Surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, and the like, and can be used in a dentifrice together with the inventive silica material. A surfactant, if present, is typically used in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents can comprise mixtures of aldehydes, ketones, esters, phenols, acids, and aliphatic, aromatic and other alcohols.

In addition, colorants can be added to improve the aesthetic appearance of the dentifrice product. Suitable colorants are selected from colorants approved by appropriate regulatory bodies such as the FDA and those listed in the European Food and Pharmaceutical Directives and include pigments, such as TiO₂, and colors such as FD&C and D&C dyes.

Thickening agents can, in various aspects, be useful in the dentifrice compositions of the present invention to provide a gelatinous structure that stabilizes the toothpaste against phase separation. Exemplary thickening agents can include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; as well as mixtures of these compounds. Typical levels of thickening agents or binders can, in various aspects, range from about 0 wt. % to about 15 wt. % of a dentifrice composition.

Therapeutic agents are optionally used in the compositions of the present invention to provide for the prevention and treatment of dental caries, periodontal disease and temperature sensitivity. Examples of therapeutic agents, without intending to be limiting, are fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents can be used in dentifrice formulations singly or in combination at a therapeutically safe and effective level.

In another aspect, preservatives can also be optionally added to the compositions of the present invention to prevent bacterial growth. Suitable preservatives approved for use in oral compositions such as methylparaben, propylparaben and sodium benzoate, or combinations thereof, may be added in safe and effective amounts.

The dentifrices disclosed herein can also a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like. Water can be used in a dentifrice composition to balance the composition, for example, from about 0 wt. % to about 60 wt. %, and provide desirable rheological properties.

In still another aspect, the inventive heat treated precipitated silica material can exhibit a refractive index approximately equal to that of the remaining dentifrice composition. In one aspect, such a refractive index can improve the clarity of the resulting dentifrice composition.

In another aspect, the inventive heat treated silica materials can provide desirable PCR and/or RDA values when incorporated into a dentifrice comprising a PVP/H₂O₂ complex. For example, a precipitated silica material heat treated at 750° C. (see Sample 5 in the Examples) can exhibit a PCR value of 101 and an RDA value of 116, when incorporated into such a dentifrice composition.

### Stability

As noted above, the inventive heat treated precipitated silica material can exhibit improved compatibility with a whitening component, such as, for example, a polyvinylpyrrolidone-hydrogen peroxide composition. Such improved compatibility can result in improved stability in, for example, consumer packaging. In one aspect, a dentifrice comprising the inventive heat treated precipitated silica material can exhibit improved tube stability at 60° C. by reducing or eliminating decomposition of the peroxide containing components. In various aspects, tube stability can be improved from less than about 1 day to a period of from about 7 to about 28 days, for example, about 7, 8, 9, 10, 11, 12, 13, 14, 16, 18, 20, 22, 24, 26, or 28 days. In still other aspects, tube stability can be improved from less than about 1 day to up to about 84 days or greater, for example, 50, 55, 60, 65, 70, 75, 80, or 84 days. For example, in one aspect, the dentifrice composition comprising the heat treated precipitated silica material can exhibit a tube stability at 60° C. of at least about 5 days, at least about 7 days, or at least about 10 days, while in another aspect, the dentifrice composition comprising the heat treated precipitated silica material can exhibit a tube stability at 60° C. of from about 5 days to about 40 days, from about 7 days to about 50 days, or from about 10 days to about 60 days. Stability at 60° C. is predictive of long term stability as observed at ambient conditions.

The present invention can be further described in a number of exemplary and non-limiting aspects, as provided below.

Aspect 1: A dentifrice comprising a precipitated silica material, wherein the precipitated silica material has been heated at a temperature and for a period of time sufficient to remove all or a portion of water from the surface thereof.

Aspect 2: A dentifrice comprising a polyvinylpyrrolidone-hydrogen peroxide complex and a precipitated silica material, wherein the precipitated silica material has been heated at a temperature and for a period of time sufficient to at least partially dehydroxylate the surface thereof.

Aspect 3: A dentifrice comprising a polyvinylpyrrolidone-hydrogen peroxide complex and a precipitated silica material, wherein the precipitated silica material has no or substantially no free or chemically bound water on a surface thereof.

Aspect 4: The dentifrice of any of aspects 1-3, wherein the precipitated silica material has a lower surface concentration of hydroxyl groups as compared to a conventional precipitated silica material.

Aspect 5: The dentifrice composition of any of aspects 1-3, wherein a portion of the precipitated silica material is crystalline.

Aspect 6: The dentifrice composition of any of aspects 1-3, wherein no more than 0.1 wt. % of the precipitated silica material comprises cristobalite.

Aspect 7: The dentifrice composition of any of aspects 1-3, wherein the precipitated silica material exhibits a loss on ignition of less than about 2 wt. %.

Aspect 8: The dentifrice composition of any of aspects 1-3, having improved stability as compared to a dentifrice comprising a polyvinylpyrrolidone-hydrogen peroxide complex and a conventional non-heat treated precipitated silica material.

Aspect 9: The dentifrice composition of any of aspects 1-3, having a tube stability at 60° C. of at least about 14 days.

Aspect 10: The dentifrice composition of any of aspects 1-3, having a tube stability at 60° C. of at least about 21 days.

Aspect 11: The dentifrice composition of any of aspects 1-3, having a tube stability at 60° C. of at least about 28 days.

Aspect 12: A method for preparing a dentifrice composition, the method comprising heat treating a precipitated silica material at a temperature and for a period of time sufficient to: a) remove all or a portion of water from the precipitated silica material without changing the structure thereof b) at least partially dehydroxylate the surface of the precipitated silica material; or a combination thereof, and then contacting the precipitated silica material with a polyvinylpyrrolidone-hydrogen peroxide complex.

Aspect 13: The method of aspect 12, comprising heat treating the precipitated silica material at a temperature of from about 650° C. to about 1,000° C. for a period of from about 14 to about 18 hours to form a heat treated silica material.

Aspect 14: The method of aspect 12, wherein heat treating comprises heating the silica material at a temperature of from about 700° C. to about 950° C. for a period of from about 14 to about 18 hours.

Aspect 15: The method of aspect 12, wherein heat treating comprises heating the silica material at a temperature of from about 750° C. to about 800° C. for a period of from about 14 to about 18 hours.

Aspect 16: The method of aspect 12, wherein heat treating comprises heating the silica material at a temperature of about 850° C. for a period of from about 120 minutes to about 960 minutes.

Aspect 17: The method of aspect 12, wherein heat treating comprises heating the silica material at a temperature of about 950° C. for a period of from about 20 to about 240 minutes.

Aspect 18: The method of any of aspects 12-17, wherein heat treating comprises heating at a temperature and for a time such that the heat treated silica material comprises from greater than zero to less than about 0.1 wt. % cristobalite.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Preparation of Silica Materials

In a first example, an exemplary silica material was prepared as described below.

5.6 L of silicate (19.5%, 1.180 g/mL, 3.32 MR) and 13.9 L of water were added to the 30 gallon reactor and heated to 87° C. while stirring at 150 RPM. Silicate (19.5%, 1.180 g/mL, 3.32 MR) and sulfuric acid (17.1%, 1.12 g/mL) were then simultaneously added at 1.1 L/min and 0.33 L/min, respectively, for 47 minutes. After 47 minutes, the flow of silicate was stopped and the pH was adjusted to 5.5 with continued flow of acid. Once pH 5.5 was reached, the batch was allowed to digest for 10 minutes at 90° C. After digestion, the batch was filtered and washed to a conductivity of about 1500 μS and was spray dried. The batch was hammer milled to an average particle size of approximately 10 μm. It should be noted that other synthesis methods, for example, in batch, semi-continuous, and/or continuous processes, and other parameters, for example, concentrations and/or temperatures, can be used to prepare a desired precipitated silica material, and that the present disclosure is not intended to be limited to any particular synthesis method.

### Example 2

### Preparation of Silica Materials

In a second example, a silica material, similar to that prepared in Example 1 was heat treated and evaluated. The silica material was split into six parts. One portion (Sample 1) of the batch was utilized without further heat treatment. The five additional portions (Samples 2-6) were heat treated at various temperatures. A Lindberg Blue M oven (Model # BF51842C) was used to heat treat the silica materials. Silica material samples were placed in ceramic bowls in the cold oven set to the appropriate temperature. The time to reach the appropriate temperature was less than 2 hours. The samples were heat treated for 16±2 hours. Sample size for the heat treatments was varied from about 60 grams to about 650 grams. After the heating time was reached, the oven was turned off and samples were allowed to cool as the oven cooled (˜2-4 hours). Once the samples were cool enough to handle, they were placed in a desiccator or storage container to prevent moisture pick-up.

The chemical and physical properties from analysis of the precipitated silica materials (Samples 1-6) are detailed in Table 1, below. No significant change in oil absorption, the CTAB values and the BET surface area was observed for silica material treated at about 750° C. When the silica material was heat treated at a temperature of about 1000° C., a drop in oil absorption, CTAB value and BET surface area was observed. The Einlehner Abrasion values for all samples increased upon heating. In addition, the moisture corrected water absorption values decreased steadily as the heat treatment temperature increased, as detailed below.

TABLE 1
Physical properties of heat treated silica materials.

| | | Sample 2 | Sample 3 | Sample 4 | Sample 5 | Sample 6 |
|---|---|---|---|---|---|---|
| | Sample 1 | Heat | Heat | Heat | Heat | Heat |
| | No heat | treated at | treated at | treated at | treated at | treated at |
| | treatment | 150° C. | 350° C. | 550° C. | 750° C. | 1000° C. |
| % H₂O | 7.2 | 3.3 | 2.2 | 1.67 | 0.71 | 0.14 |
| % H₂O + % LOI | 12.12 | 11.09 | 9.92 | 7.3 | 1.26 | 0.05 |
| Pre-dried | 5.3 | 8.06 | 7.87 | 5.73 | 0.55 | −0.09 |
| % LOI | | | | | | |
| %325 Mesh | 0.68 | 0.83 | 0.8 | 0.7 | 0.6 | 31.7 |
| 5% pH | 7.33 | 7.5 | 7.5 | 6.64 | 6.8 | 10.51 |
| Pour Density | 13.9 | 13.6 | 13.9 | 13.9 | 14.9 | 27.1 |
| (lb/ft³) | | | | | | |
| Pack density | 26 | 24 | 25 | 25 | 26 | 44.6 |
| (lb/ft³) | | | | | | |
| % Na₂SO₄ | 1.29 | 1.53 | 1.69 | 1.61 | 1.61 | 1.84 |
| (by | | | | | | |
| conductivity) | | | | | | |
| Particle size | 9.22 | 9 | 8.71 | 8.87 | 7.87 | 12.18 |
| (Horiba) | | | | | | |
| Median | | | | | | |
| (micron) | | | | | | |
| Particle size | 12.55 | 11.98 | 11.53 | 11.81 | 10.98 | 16.58 |
| (Horiba) | | | | | | |
| Mean | | | | | | |
| (micron) | | | | | | |
| Brightness | 99.3 | 99.1 | 99.6 | 100.1 | 100.1 | 99.9 |
| (Technidyne) | | | | | | |
| Surface Area | 46 | 45 | 46 | 43 | 44 | 8 |
| (CTAB), pre- | | | | | | |
| dried (m²/g) | | | | | | |
| Surface Area | 79 | 51 | 43 | 38 | 30 | 0 |
| (BET) degass | | | | | | |
| 240° C. (m²/g) | | | | | | |
| Oil | 97 | 97 | 105 | 95 | 98 | 43 |
| Absorption | | | | | | |
| (cc/100 g) | | | | | | |
| Hg Total | 1.56 | 1.59 | 1.59 | 1.62 | 1.56 | 1.07 |
| Intruded | | | | | | |
| Volume | | | | | | |
| (cc/g) | | | | | | |
| Hg Intrusion | 0.21 | 0.23 | 0.21 | 0.24 | 0.3 | 2.81 |
| (Med. Pore | | | | | | |
| Dial (Vol.)) | | | | | | |
| Water | 110 | 117.9 | 118.1 | 111.9 | 106.1 | 77.9 |
| Absorption | | | | | | |
| (cc/100 g, as | | | | | | |
| received) | | | | | | |
| Water | 126.3 | 125.3 | 123 | 115.6 | 107.6 | 78.1 |
| Absorption | | | | | | |
| (cc/100 g, | | | | | | |
| moisture | | | | | | |
| corr.) | | | | | | |
| Brass | 2.44 | 2.73 | 3.82 | 5.18 | 6.2 | 20.47 |
| Einlehner | | | | | | |
| (BE) | | | | | | |
| Abrasion (mg | | | | | | |
| loss/100,000 | | | | | | |
| rev) | | | | | | |
| XRD* | A. | A. | A. | A.T.C. | A.T.C. | C. |
| Tube | <1 | <1 | <1 | <1 | 7 to 14 | |
| Stability | | | | | | |
| (days, 60° C.) | | | | | | |

*A.—Amorphous; A.T.C.—Amorphous with traces of Cristobalite; C.—Cristobalite

The XRD results illustrate a phase transition from an amorphous structure for non-heat treated or low temperature heat treated silica materials to an amorphous structure with traces (less than 1 wt. %) of cristobalite for silica materials treated at temperatures from about 500° C. to about 800° C., and further to a cristobalite structure for silica materials heat treated at a temperature of about 1000° C. and above.

### Example 3

### Toothpaste Tube Filling and Sealing

A toothpaste composition comprising the inventive heat treated silica material was prepared in a laboratory mixer. 37 grams of the silica based PVP/H₂O₂ complex style formula was placed in a 1 inch by 4 inch plastic-foil lined toothpaste tube, available from Pechiney Plastic Packaging Inc. (1×4 2183) (Model #2119). For comparison, 43 grams of conventional calcium pyrophosphate based toothpaste are needed to fill the same tubes, the difference being due to the density difference between the compositions. The comparative compositions are presented in Table 2, wherein the conventional calcium pyrophosphate based formula is denoted as Comparative 1, and the inventive formulation is denoted as Inventive 1. The tubes were heat sealed using a Vertrod Thermal Impulse Heat Sealing Machine (Model #4H/HTV-SP) from PAC Machinery, with heat and dwell settings both set to 10 and the compression of the heat sealing lever set at maximum. The heating time used to seal the tubes was approximately 21 seconds.

When incorporated into the dentifrice composition, the inventive heat treated silica material (Sample 5, 750° C.) exhibited a PCR of 101 and an RDA of 116.

TABLE 2
Comparative dentifrice formulations

| | Comparative 1 | Inventive 1 |
|---|---|---|
| Propylene Glycol | 25.000 | 32.477 |
| Tetrasodium Pyrophosphate | 2.000 | 2.000 |
| Sodium Saccharin | 0.600 | 0.600 |
| Sucralose | 0.050 | 0.050 |
| Phosphoric Acid | 0.200 | 0.200 |
| BHT | 0.030 | 0.030 |
| Sodium Monofluorophosphate | 0.760 | 0.760 |
| Crosslinked PVP/hydrogen peroxide | 5.500 | 5.500 |
| PEG-12 | 10.000 | 12.996 |
| PEG/PPG 116/66 Copolymer | 10.000 | 12.996 |
| Glycerin | 5.110 | 6.641 |
| Fumed Silica | 1.500 | 1.500 |
| Calcium Pyrophosphate | 35.000 | — |
| Silica Abrasive | — | 20.000 |
| Flavor | 2.250 | 2.250 |
| SLS | 2.000 | 2.000 |
| Total | 100.000 | 100.000 |

Stability of the resulting tubes (60° C.) increased from less than 1 day for silica materials heat treated at lower temperatures to about one to two weeks for the inventive silica material heat treated at about 750° C. While not wishing to be bound by theory, the increase in the tube stability can be explained by the dehydroxylation of the silica surface. It is speculated that silica particles having a large number of hydroxyl groups can catalyze the decomposition of the polyvinylpyrrolidone/hydrogen peroxide (PVP/H₂O₂) complex by interfering with the hydrogen bonding between PVP and H₂O₂. The heat treatment at elevated temperatures decreases the amount of free and chemically bound water on silica particles, and thus, decreases the decomposition rate of PVP/H₂O₂ complex and as a result increases the tube stability.

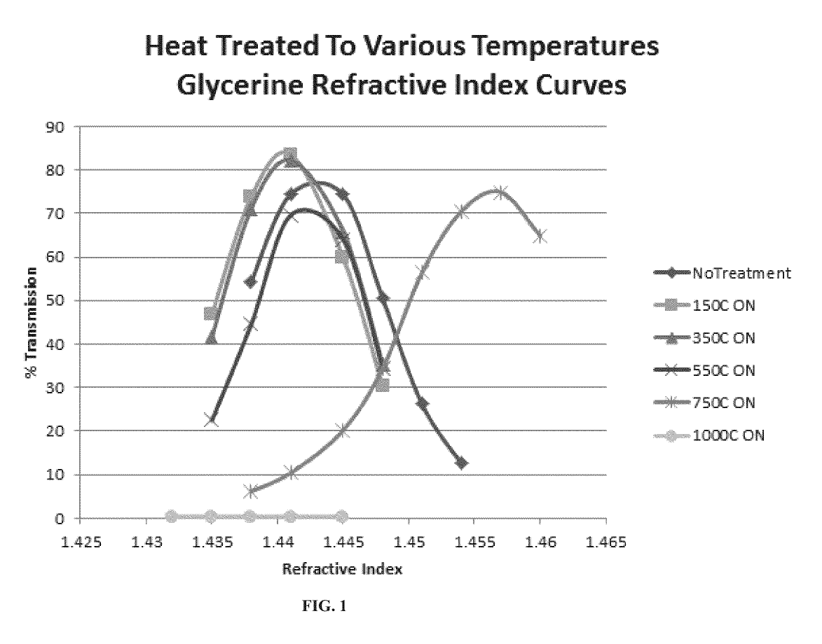

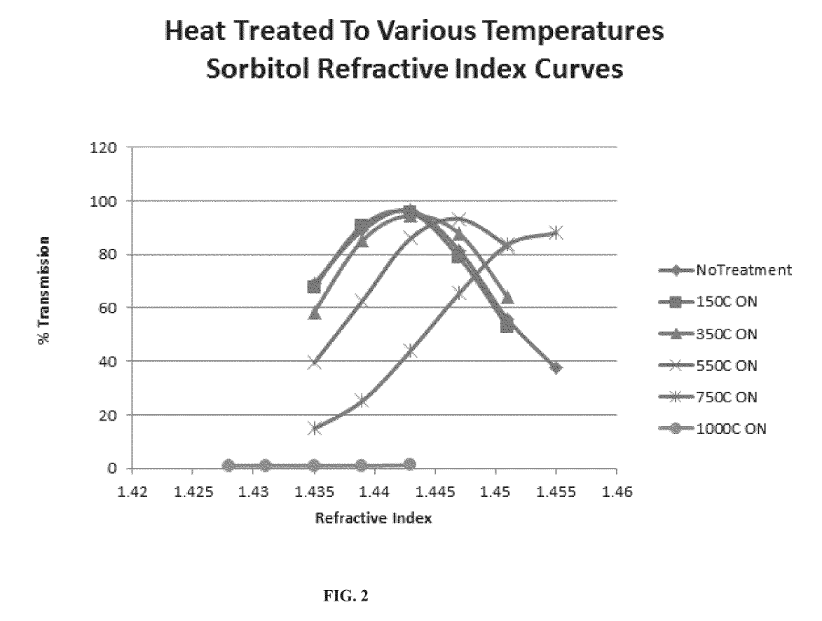

It was demonstrated that in addition to improved tube stability heat treated at the higher temperatures, silica materials have higher refractive index values than those that are either not heat treated or heat treated at lower temperatures. The percentage of transmission measured at sorbitol and glycerin refractive indices for silica materials that were heat treated at various temperatures is summarized in Tables 3 and 4, below. FIGS. 1-2 illustrate the refractive indices curves at various temperatures. As one of ordinary skill in the art would readily appreciate, silica materials having higher refractive indices yield improved clarity for the final anhydrous dentifrice formulations. Surprisingly, the peak transmittance in glycerine was at a refractive index greater than 1.45 (in the 1.45-1.46 range), and the peak transmittance in sorbitol was at a refractive index greater than 1.45 (in the 1.45-1.46 range).

The refractive index of an exemplary dentifrice formulation was tested without the inventive heat treated silica and determined using a “Leica Auto Abbe Refractometer Model #10500-802 to be 1.4505. The % transmission maximums for Sample 5 (750° C. heat treatment) in glycerine and sorbitol were determined to be 1.457 and 1.455, respectively, indicating a close match. In one aspect, such a match can provide improved toothpaste clarity.

TABLE 3
% Transmittance at glycerin refractive indices.

| | | Sample 2 | Sample 3 | Sample 4 | Sample 5 | Sample 6 |
|---|---|---|---|---|---|---|
| Refractive | Sample 1 | Heat | Heat | Heat | Heat | Heat |
| Index | No heat | treated | treated | treated | treated | treated |
| (glycerin) | treatment | at 150° C. | at 350° C. | at 550° C. | at 750° C. | at 1000° C. |
| 1.429 | | | | | | |
| 1.432 | | | | | | 0.4 |
| 1.435 | | 46.8 | 41.6 | 22.5 | | 0.4 |
| 1.438 | 54.3 | 73.9 | 71.1 | 44.6 | 6.2 | 0.4 |
| 1.441 | 74.4 | 83.6 | 82.2 | 69.6 | 10.4 | 0.4 |
| 1.445 | 74.3 | 59.8 | 65.9 | 64 | 20.2 | 0.4 |
| 1.448 | 50.5 | 30.4 | 35.4 | 34.4 | 34.4 | |
| 1.451 | 26.2 | | | | 56.4 | |
| 1.454 | 12.6 | | | | 70.5 | |
| 1.457 | | | | | 74.9 | |
| 1.46 | | | | | 64.9 | |

TABLE 4
% Transmittance at sorbitol refractive indices.

| | | Sample 2 | Sample 3 | Sample 4 | Sample 5 | Sample 6 |
|---|---|---|---|---|---|---|
| Refractive | Sample 1 | Heat | Heat | Heat | Heat | Heat |
| Index | No heat | treated | treated | treated | treated | treated |
| (sorbitol) | treatment | at 150° C. | at 350° C. | at 550° C. | at 750° C. | at 1000° C. |
| 1.422 | | | | | | |
| 1.428 | | | | | | 1 |
| 1.431 | | | | | | 1 |
| 1.435 | 69 | 67.6 | 58.4 | 39.4 | 14.8 | 1 |
| 1.439 | 89 | 90.8 | 85.2 | 62.6 | 25.2 | 1 |
| 1.443 | 96.4 | 95.6 | 94.6 | 86 | 44 | 1.2 |
| 1.447 | 81.4 | 78.8 | 87.7 | 93.3 | 65.4 | |
| 1.451 | 55.8 | 52.8 | 64 | 83 | 83.6 | |
| 1.455 | 37.4 | | | | 88 | |

Table 5 demonstrates the elemental content and the change in the amount of free and bound water for the silica materials that were not heat treated (Sample 1) and heat treated at about 750° C. (Sample 5).

TABLE 5
Elemental content of silica materials.

| | |
|---|---|
| Sample 1 | Sample 5 |
| No heat treatment | Heat treated at 750° C. |

| | | |
|---|---|---|
| % LOI | 5.72% | 0.89% |
| % Moisture | 6.93% | 0.47% |
| Al₂O₃ | 0.21% | 0.22% |

| | | | | |
|---|---|---|---|---|
| CaO | 25 | ppm | 25 | ppm |
| Co | 0.1 | ppm | 0.1 | ppm |
| Cr | 2.1 | ppm | 2.2 | ppm |
| Cu | 1.0 | ppm | 1.1 | ppm |
| Fe₂O₃ | 321 | ppm | 335 | ppm |
| MgO | 33 | ppm | 35 | ppm |
| Mn | 0.5 | ppm | 0.6 | ppm |
| Mo | <0.1 | ppm | <0.1 | ppm |

| | | |
|---|---|---|
| Na₂O | 1.23% | 1.25% |

| | | | | |
|---|---|---|---|---|
| Ni | 0.5 | ppm | 0.5 | ppm |
| TiO₂ | 102 | ppm | 106 | ppm |
| V | 0.7 | ppm | 0.8 | ppm |

### Example 4

### Addition of Water to Dentifrice Containing Heat Treated Silica

In a fourth example, 16.7 wt. % water was added to a fumed silica sample and then added to the formulation described in Table 2 at a 1.5 wt. % loading level with 20% calcium pyrophosphate. The resulting dentifrice exhibited a tube stability (60° C.) of about 28 days. Similarly, calcium pyrophosphate with 16.7 wt. % added water was incorporated into the formulation detailed in Table 2. The resulting dentifrice exhibited a tube stability (60° C.) of about 28 days, indicating that decomposition of the PVP/H₂O₂ complex with non-heat treated silica materials is not due solely to the addition of water. Thus the surface activity (e.g., free moisture and hydroxyl group concentration) of a silica material (not just water content) can affect the stability of a PVP/H₂O₂ complex.

### Example 5

### Tube Stability at Various Heat Treatment Conditions

In a fifth example, a series of silica materials were heat treated at 750° C., 850° C., and 950° C., for periods of time ranging from 1 minute to 960 minutes. The heat treated silica materials were formulated into a dentifrice composition and a toothpaste tube was filled with the composition. The length of time for which the toothpaste tube remained stable was then determined. The stability of a control sample comprising a calcium pyrophosphate dentifrice material (i.e., not including the inventive heat treated silica material) was also measured.

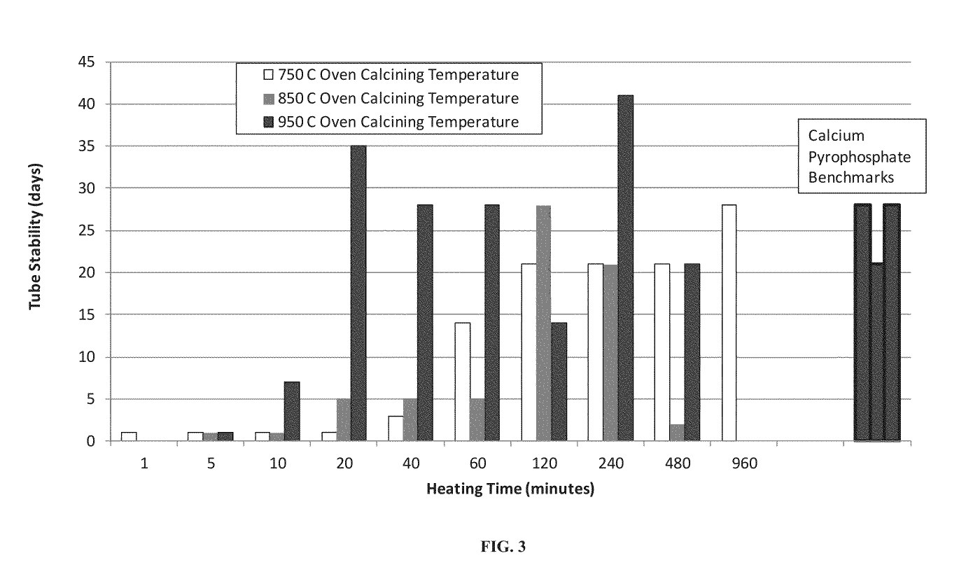

As illustrated in FIG. 3, when heat treated at 750° C. for 16 hours, the tube stability of the dentifrice containing the inventive heat treated silica was approximately equal to the control calcium pyrophosphate sample. When heat treated at 850° C. for 120 or 240 minutes, the tube stability of the dentifrices containing the inventive heat treated silicas were equal to or greater than the control sample. When heat treated at 950° C. for 20, 40, 60, or 240 minutes, the tube stability of the dentifrices containing the inventive heat treated silicas were equal to or greater than the control calcium pyrophosphate sample. For comparative purposes, a non heat treated silica sample would result in a tube stability value of less than one day.

What is claimed is:

1. A dentifrice composition comprising a polyvinylpyrrolidone-hydrogen peroxide complex and a precipitated silica material, wherein:
the precipitated silica material comprises from greater than 0 to less than about 1 wt. % cristobalite; and
the precipitated silica material is characterized by a loss on ignition of less than about 2 wt. %.

2. The composition of claim 1, wherein the precipitated silica material comprises from greater than 0 to less than about 0.1 wt. % cristobalite.

3. The composition of claim 1, wherein the precipitated silica material is characterized by a loss on ignition of less than about 1 wt. %.

4. The composition of claim 1, wherein the precipitated silica material is further characterized by:
a BET surface area in a range from about 20 to about 60 m²/g;
an oil absorption in a range from about 70 to about 120 cc/100 g; and
a Brass Einlehner abrasion value in a range from about 4 to about 20 mg loss/100,000 revolutions.

5. The composition of claim 1, wherein the precipitated silica material is further characterized by a Brass Einlehner abrasion value in a range from about 5 to about 10 mg loss/100,000 revolutions.

6. The composition of claim 1, wherein the precipitated silica material is further characterized by:
a peak transmittance in glycerine at a refractive index greater than 1.45; and/or
a peak transmittance in sorbitol at a refractive index greater than 1.45.

7. The composition of claim 1, wherein the precipitated silica material is further characterized by:
a peak transmittance in glycerine at a refractive index in a range from about 1.45 to about 1.46; and
a peak transmittance in sorbitol at a refractive index in a range from about 1.45 to about 1.46.

8. The dentifrice composition of claim 1, having a tube stability at 60° C. of at least about 5 days.

9. The dentifrice composition of claim 1, having a tube stability at 60° C. in a range from about 7 days to about 50 days.

10. A method for preparing a dentifrice composition, the method comprising:
(a) heat treating a precipitated silica material at a temperature and for a period of time sufficient to form a heat treated silica material comprising from greater than 0 to less than about 1 wt. % cristobalite, the heat treated silica material characterized by a loss on ignition of less than about 2 wt. %; and
(b) contacting the heat treated silica material with a polyvinylpyrrolidone-hydrogen peroxide complex and other ingredients to form the dentifrice composition.

11. The method of claim 10, wherein the heat treated silica material comprises from greater than 0 to less than about 0.1 wt. % cristobalite, and is characterized by a loss on ignition of less than about 1 wt. %.

12. The method of claim 10, wherein the method comprises heat treating the precipitated silica material at a temperature of from about 650° C. to about 1000° C. for a period of time from about 20 minutes to about 20 hours to form the heat treated silica material.

13. The method of claim 10, wherein the method comprises heat treating the precipitated silica material at a temperature of from about 700° C. to about 950° C. for a period of time from about 14 to about 18 hours to form the heat treated silica material.

14. The method of claim 10, wherein the dentifrice composition has a tube stability at 60° C. of at least about 10 days.

15. The method of claim 10, wherein the dentifrice composition has a tube stability at 60° C. in a range from about 5 days to about 40 days.

16. The method of claim 10, wherein the dentifrice composition has a tube stability at 60° C. that is greater than a tube stability at 60° C. of a dentifrice composition containing the precipitated silica material.

17. The method of claim 10, wherein the dentifrice composition has a tube stability at 60° C. that is at least 5 times greater than a tube stability at 60° C. of a dentifrice composition containing the precipitated silica material.

18. A precipitated silica material comprising from greater than 0 to less than about 1 wt. % cristobalite, and characterized by a loss on ignition of less than about 2 wt. %.

19. The precipitated silica material of claim 18, wherein the precipitated silica material comprises from greater than 0 to less than about 0.1 wt. % cristobalite, and wherein the precipitated silica material is characterized by a loss on ignition of less than about 1 wt. %.

20. The precipitated silica material of claim 18, wherein the precipitated silica material is further characterized by:
a BET surface area in a range from about 20 to about 60 m²/g;
an oil absorption in a range from about 70 to about 120 cc/100 g;
a Brass Einlehner abrasion value in a range from about 4 to about 20 mg loss/100,000 revolutions;
a peak transmittance in glycerine at a refractive index in a range from about 1.45 to about 1.46; or
a peak transmittance in sorbitol at a refractive index in a range from about 1.45 to about 1.46; or
any combination thereof.
